(19) United States
(12) Patent Application Publication
(10) Pub. No.: US 2008/0132970 A1
(43) Pub. Date: Jun. 5, 2008

(54) METHOD AND SYSTEM FOR TREATMENT OF INTRACTABLE SCROTAL AND/OR TESTICULAR PAIN

(76) Inventor: Giancarlo Barolat, Golden, CO (US)

Correspondence Address: SHERIDAN ROSS PC, 1560 BROADWAY, SUITE 1200, DENVER, CO, 80202, US

(21) Appl. No.: 11/567,136

(22) Filed: Dec. 5, 2006

(51) Int. Cl. A61N 1/00; A61F 7/00; A61B 19/00

(52) U.S. Cl. 607/46; 607/152; 607/99; 606/129

(57) ABSTRACT

A system and method is provided for the implantation and treatment of pain, such as scrotal and/or testicular pain, using electrical stimulation. The implanted portion of the system includes an implantable pulse generator that provides an electrical current through an implantable lead that extends to at least one electrode or an electrode array. The electrode is implanted into the relatively fragile tissue of the scrotal sac using a method that eliminates or otherwise minimizes damage to the tissue of the scrotal sac during the implantation procedure. An embodiment of the invention allows in-place testing of the electrode while the sheath still covers the implantable lead.

### FIELD

The present invention is related to the electrical stimulation of nerves, and more particularly, to the electrical stimulation of nerves within the scrotal sac for the treatment of intractable scrotal pain and/or testicular pain.

### BACKGROUND

Intractable pain is a condition that some patients endure because of a variety of reasons, including disease and injury. With regard to the present application, some men suffer from chronic testicular pain and/or intractable scrotal pain because of disease and/or because of a severe injury to the testicles. In some instances, the injury has required the removal of one or both of the testicles. Despite removal of one or both of the testicles, some men still experience severe pain that is difficult to treat.

Although some prescribed drugs can be helpful for addressing chronic pain, certain individuals can have difficulties with various side effects associated with pain relieving drugs. Accordingly, in some instances, electrical stimulation of nerves within the body is used to control pain of a patient suffering from intractable pain. In general, electrical stimulation is achieved by implanting a pulse generator with a lead and one or more electrodes that deliver electrical stimulation to the target nerves that are associated with the pain.

Although electrical stimulation has been used to treat pain within various parts of the body, including the back and legs, electrical stimulation requires the implantation of the electrodes in the vicinity of the nerves associated with the pain. For men suffering from intractable scrotal and/or testicular pain, this would require implantation of an electrode in the vicinity of the scrotal sac, and this can be problematic because of the relatively delicate structure of the tissue in the scrotum.

As further background to existing treatment systems, reference is made to U.S. Patent Application Publication No. 2005/0010259 that discloses electrical stimulation and drug therapy systems. The content of the aforementioned application is incorporated herein by reference in its entirety. Although the '259 application mentions use of implantable devices to treat various disorders, it does not disclose how to implant a treatment device in the scrotal sac.

Thus, one problem associated with placement of one or more electrodes to treat intractable scrotal and/or testicular pain is that electrodes need to be implanted into the scrotal sac, which requires an incision, and then subsequent placement of the electrodes through the incision and into the scrotal sac. For treatment of intractable scrotal and/or testicular pain, therefore, it would be advantageous if a method existed that allowed a surgeon to implant one or more electrodes directly into the scrotal sac despite the relatively delicate nature of the tissue within the scrotum.

### SUMMARY

The present invention addresses the shortcomings of the prior art by providing a method and system for the treatment of pain, particularly intractable scrotal and/or testicular pain. The system used generally comprises an implatable pulse generator, a lead, and one or more electrodes. The one or more electrodes are carefully implanted into the desired tissue (e.g., scrotal sac) utilizing a blunt-tipped sheath to allow insertion of the electrode into the scrotal sac. After implantation of the electrical stimulation system, the system is used to provide an electrical signal to the targeted nerves, wherein the electrical signal modifies the pain signal received by the patient's brain. Although different patients react differently to electrical stimulation treatment, in successful treatments the patient perceives a sensation that is not pain, but rather, may be a tingling sensation instead of pain.

Although the explanation of the devices and the methods for treatment are described for the treatment of pain in the scrotal area, it will be appreciated by those skilled in the art that the devices and methods presented herein are applicable for the treatment of pain in other areas of the body other than the scrotum of a man. It is also to be understood that the present invention includes a variety of different versions or embodiments, and this Summary is not meant to be limiting or all inclusive. This Summary provides some general descriptions of some of the embodiments, but may also include some more specific descriptions of certain embodiments.

In accordance with embodiments of the present invention, the surgeon first makes an incision in the pubic area or lower abdomen for allowing access to the scrotal sac internally, such as under the skin or subcutaneously. From an anatomically superior position, the surgeon preferably utilizes a blunt-tipped sheath with a flexible rod or introducer positioned in its longitudinal bore to insert the sheath into the scrotal sac. The blunt-tipped sheath is shaped to allow its distal end to push into and/or separate the relatively delicate tissue of the scrotal sac without damaging the tissue within the scrotal sac. Rod or introducer acts as a stiffener to all the distal end of the sheath to separate the tissue without bending excessively. The rod or introducer is then withdrawn and the electrode or electrode array is inserted into the hollow bore of the sheath. Accordingly, the sheath is sized for holding the electrode or electrode array that is connected to an electrical lead.

Depending upon the stiffness of the sheath, the sheath and electrode may be inserted into the scrotal sac without first using an introducer. Thus, in accordance with embodiments of the present invention, the surgeon first makes an incision in the pubic area or lower abdomen for allowing access to the scrotal sac internally, such as under the skin or subcutaneously. From an anatomically superior position, the surgeon preferably utilizes a blunt-tipped sheath to insert the electrode or electrode array into the scrotal sac. The blunt-tipped sheath is shaped to allow its distal end to push into and/or separate the relatively delicate tissue of the scrotal sac without damaging the tissue within the scrotal sac. The sheath is sized for holding the electrode or electrode array that is connected to an electrical lead. Without the use of the blunt-tipped sheath, the electrical lead with the electrode or electrode array at its end is typically too pliable for inserting into the tissue of the scrotal sac. In addition, the electrode and electrical lead may be too narrow for direct insertion into the scrotum without damaging the tissue of the scrotum. With the electrical lead and electrode residing within the sheath, the sheath is effectively plugged, thereby preventing tissue from entering the sheath as the sheath in introduced and advanced through the tissue of the patient. Embodiments of the present invention may comprise a marking, projection or clip for assisting the surgeon in maintaining the proper relative position between the lead and the sheath.

Embodiments of the present invention include a blunt-tipped sheath that has openings along its longitudinal length that substantially correspond to the electrode or electrode array locations. Thus, during the surgical procedure, the sheath structure allows for testing of the effectiveness of the electrical stimulation while the sheath is still in place around the electrode or electrode array. More particularly, during the actual implantation surgery, the patient can be asked whether the electrical stimulation is effective to address the pain. If the location of the electrode is satisfactory, the sheath can be removed leaving the electrode/electrode array and electrical lead in place. If the initial testing is not effective, the surgeon can adjust the location of the electrode in an attempt to improve the effectiveness of the electrical stimulation in addressing the patient's pain. Once an effective implant location is found, the sheath is then removed from the scrotal sac and the incision, leaving the electrical lead and the one or more electrodes implanted in the patient.

Various embodiments of the present invention are set forth in the attached figures and in the detailed description of the invention as provided herein and as embodied by the claims. It should be understood, however, that this Summary does not contain all of the aspects and embodiments of the present invention, is not meant to be limiting or restrictive in any manner, and that the invention as disclosed herein is and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

FIG. 1 is a front elevation view of a human male with an embodiment of the implantable portion of the present invention;

FIG. 2 is an elevation view of the implantable electrical stimulation system shown in FIG. 1;

FIG. 3 is a side elevation view of a sheath in accordance with embodiments of the present invention;

FIG. 4A is a side elevation view of an electrode lead in accordance with embodiments of the present invention;

FIG. 4B is a side elevation view of a clamp in accordance with embodiments of the present invention;

FIG. 5A is a side elevation view of the devices of FIGS. 3 and 4A, wherein the device of FIG. 4A has been inserted into the device of FIG. 3;

FIG. 5B is a detail cross-section view of the combined distal ends of the devices shown in FIG. 5A;

FIG. 6 is a perspective view of devices in accordance with an embodiment of the present invention;

FIG. 7 is a front elevation view of a human male showing insertion of devices in accordance with embodiments of the present invention;

FIG. 8 is a side profile view a human male and the combined components of the implantation system of FIG. 5A;

FIG. 9 is a front elevation view of a human male in which the sheath is being removed leaving the electrical lead with one or more electrodes positioned in the scrotal sac;

FIG. 10 is side profile of a human male as shown in FIG. 9;

FIG. 11 is a side profile of a human male with the stimulation system implanted;

FIG. 12 shows a flow diagram according to an embodiment of a method of the present invention;

FIG. 13 is a front elevation view of a human male showing insertion of devices in accordance with embodiments of the present invention;

FIG. 14 is a front elevation view of a human male in which the introducer is being removed leaving the distal end of the sheath positioned within the scrotal sac;

FIG. 15 is a front elevation view of a human male in which lead is being inserted into the sheath; and

FIG. 16 shows a flow diagram according to an embodiment of a method of the present invention.

The drawings are not necessarily to scale.

### DETAILED DESCRIPTION

In accordance with embodiments of the present invention, a device and method is provided for the implantation of a treatment system for the treatment of pain, particularly intractable scrotal and/or testicular pain, using electrical stimulation. The implanted portion of the system includes an implantable pulse generator that provides an electrical current through an implantable lead that extends to at least one electrode or an electrode array. In one configuration, the electrode is implanted into the relatively fragile tissue of the scrotal sac using an apparatus and method that eliminates or otherwise advantageously mitigates damage to the tissue of the scrotal sac during the implantation procedure. The placement device also includes advantageous structural features allowing in-place testing of the electrical stimulation system while the placement device is still co-located with structure of the electrical stimulation system.

It will be appreciated by those skilled in the art that the devices and methods of the present invention have application to treatment of pain in areas of the body other than the scrotum. For example, the devices of the present invention may be used to implant an electrode in or near the spinal column or brain.

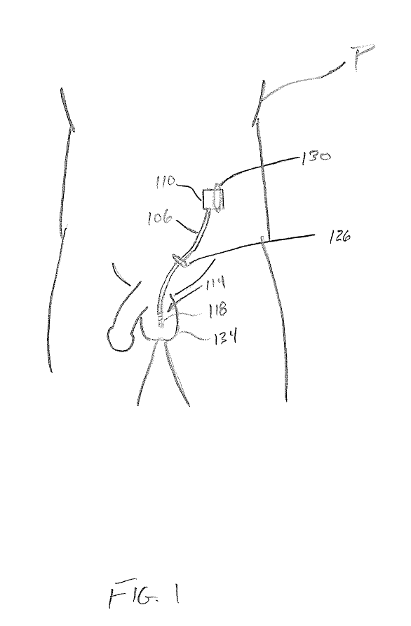

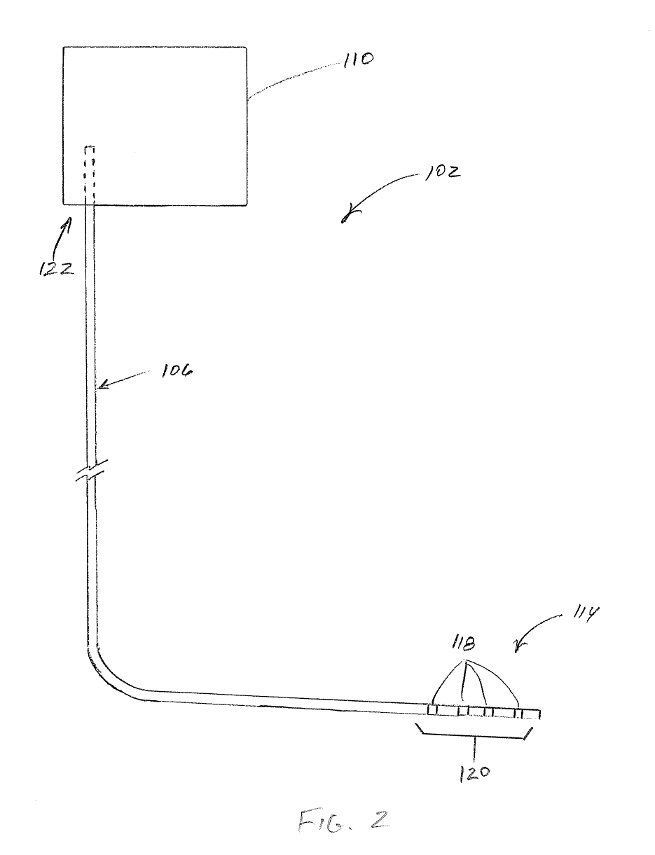

Referring now to FIG. 1, a patient P is shown with an implanted neurostimulator 102 for the treatment of scrotal and/or testicular pain. As best seen in FIG. 2, the neurostimulator 102 includes a lead 106 (also referred to herein as an “implantable lead,” a “electrode lead” and “electrical lead”) and a pulse generator 110. The lead 106 has a distal region 114 that typically comprises at least one, and more preferably, a plurality of electrodes 118, such as an electrode array 120. The proximal end 122 of the lead 106 is adapted for mating or cooperating with the pulse generator 110, an may include contacts 124 for electrical communication between the lead 106 and the pulse generator 110. The pulse generator 110, lead 106 and electrodes 118 can be permanently implanted within the patient P.

Referring again to FIG. 1, the implantation of neurostimulator 102 involves the making of at least a first incision 126 in the patient P, where the first incision 126 is located in an anatomically superior location relative to the scrotum or scrotal sac 134 of the patient P. Thus, embodiments of the present invention include a method of implanting an electrode into a human male's scrotum, where an incision is not made into the scrotum. In addition, the implantation procedure typically also involves the making of a second incision 130, where the first incision 126 is for implanting the electrode 118 and the second incision 130 is for implanting the pulse generator 110. Although other locations are possible, the second incision 130 is typically located in the abdominal region of the patient P. It is noted that the procedure could be performed by making only one incision, but that, depending upon the physical dimensions of the patient and the size of the currently available implant components, the preferred procedure typically includes more than one incision.

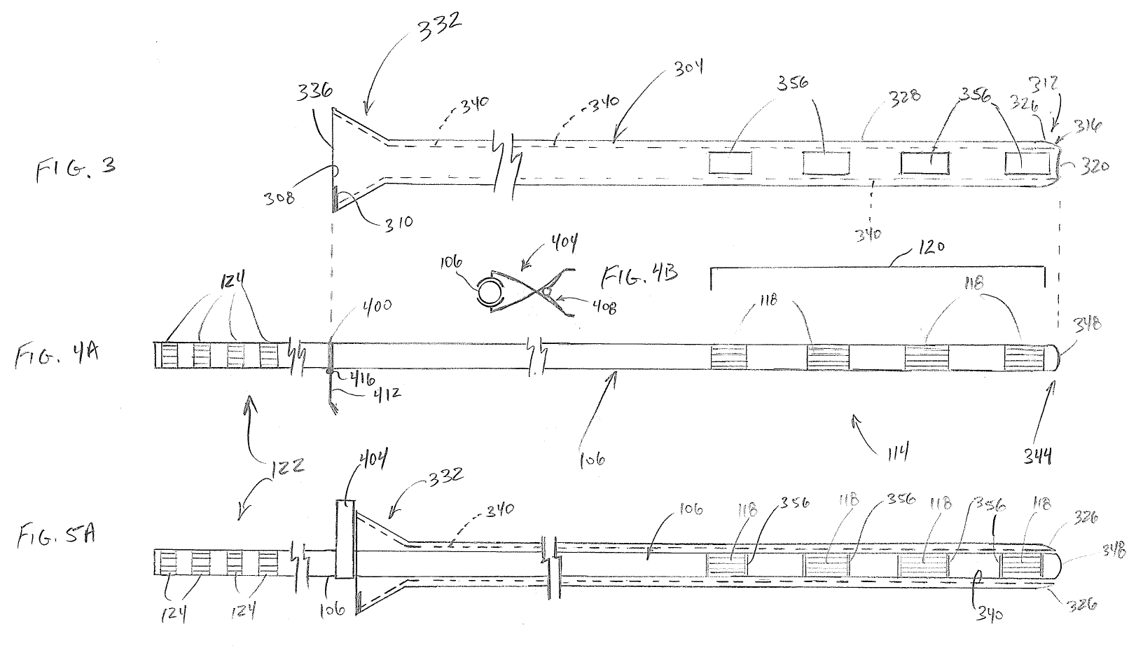

Referring now to FIGS. 3-5A, the implantation device preferably comprises a sheath 304 that is used to install the lead 106 with electrodes 118 directly into the scrotal sac 134. However, as discussed in detail below, and in accordance with embodiments of the present invention, a rod or introducer may be used with the sheath 304 to allow advancement of the sheath within more resistant tissue. The sheath 304 is preferably not rigid, but may comprise a less flexible structure than the lead 106. At least a portion of the implantation procedure is preferably preformed subcutaneously from the location of the first incision 126. To facilitate implantation of the lead 106 below the skin of the patient P, the sheath 304 includes a distal end 312 having a substantially blunt tip 316 that further includes a distal aperture 320. More particularly, the blunt tip 316 of the sheath 304 is configured for being inserted, in combination with the lead 106, directly into the tissue of the scrotal sac 134 without damaging the tissue of scrotal sac 134. The lead 106 is preferably flexible and includes a distal end 344 with a rounded or blunt end 348. The blunt end 348 of the lead 106 cooperates with the rounded distal surface 324 and sloped portion 326 of the sheath 304 to allow the lead 106 to be introduced into the rather delicate tissue of the scrotum 134 without damaging the tissue. A longitudinal bore 340 extends between the distal aperture 320 at the distal end 312 of the sheath 304, to a proximal aperture 336 at the proximal end 332 of the sheath 304. The proximal aperture 336 may be larger in size than the distal aperture 320 to facilitate easier insertion of the distal end 344 of the lead 106 into the sheath 304.

As best seen in FIG. 5B, the blunt tip 316 of the sheath 304 preferably includes a rounded distal surface 324 for deflecting the tissue of the scrotal sac 134 during insertion of the sheath 304 into the scrotal sac 134. The distal end 312 of sheath 304 may include a sloped portion 326 leading to a primary section 328. The sloped portioned 326 transitions the distal end 312 of the sheath 304 from the smaller diameter of the lead 106 to the outer and larger diameter of the sheath 304.

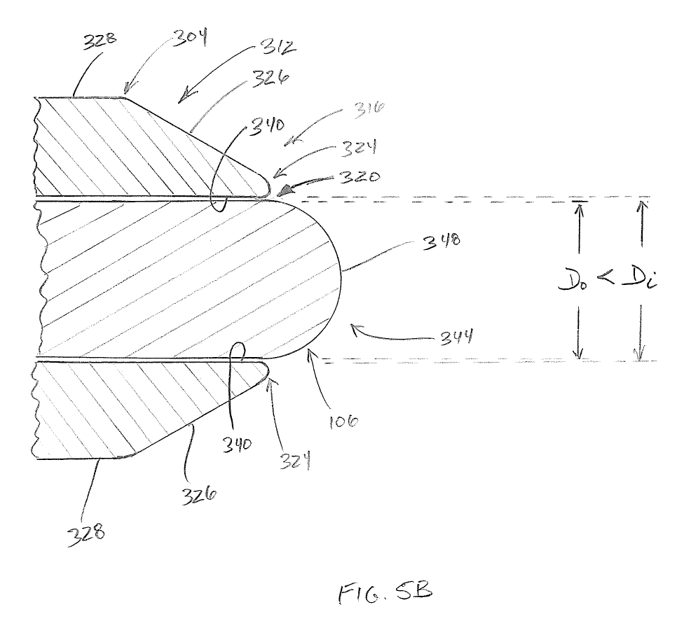

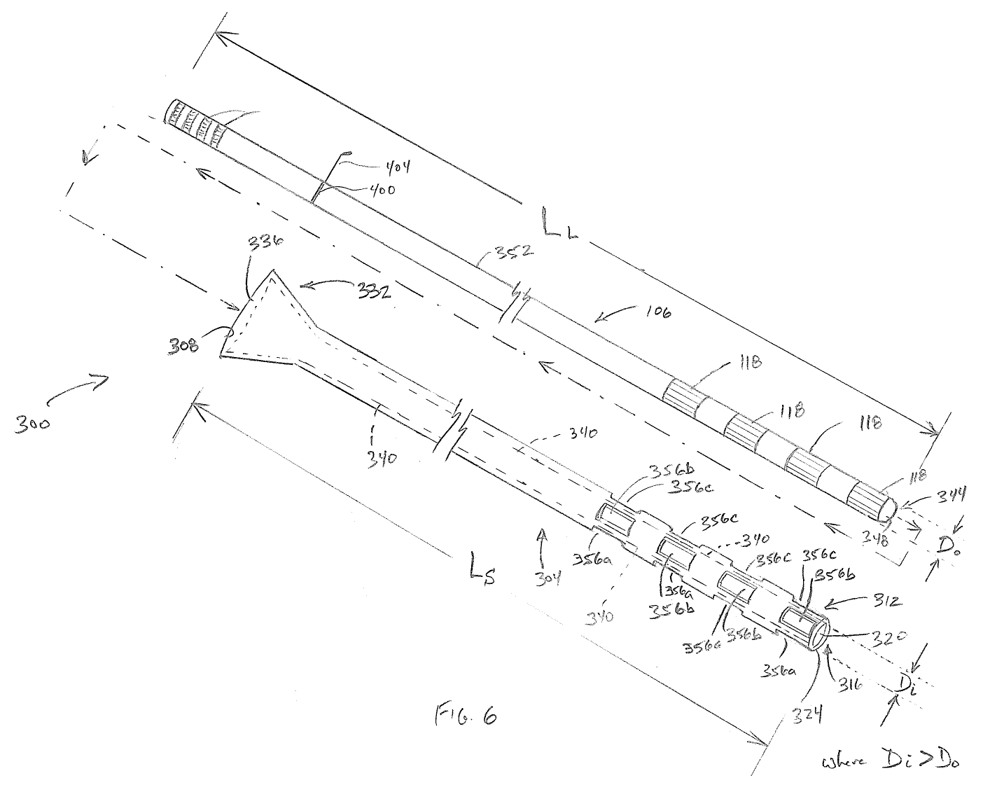

Referring still to FIGS. 4A and 5A, FIG. 4A depicts the lead 106, and FIG. 5A depicts the lead residing within the sheath 304. Accordingly, the lead 106 and the sheath 304 are sized for being axially co-located so that the lead 106 can be placed within the sheath 304, as shown in FIG. 5A.

Referring to now to FIG. 6, a perspective view of both the lead 106 and the sheath 304 are shown prior to the lead 106 being loaded into the sheath 304. Since the outer surface 352 of the lead 106 is sized for being received within the longitudinally oriented bore 340 of the sheath 304, the lead 106 has an outer diameter Do that is less than an inner diameter Di of the sheath 304. In addition, since the proximal end 122 of the lead 106 is preferably accessible after the lead 106 is inserted into the sheath 304, the lead 106 preferably comprises a lead length L_{L}, that is greater than a sheath length L,s of the sheath 304.

In accordance with embodiments of the present invention, the outer diameter of the sheath 304 is preferably between about 3 to 6 mm, and the sheath 304 preferably has a length of greater than about 15 cm. In addition, the outer diameter Do of the lead 106 is between about 2 to 4 mm, and the lead 106 preferably has a length of greater than about 20 cm. The values given above are preferred values or ranges for utilizing a sheath and lead to implant into an adult man of average size. However, these values may vary, for example, when treating a relatively small boy or a large man, or when using the sheath to implant an electrode or electrode array in another area of the body, such as the spine.

In a separate aspect of the invention, and as shown in FIG. 4, the lead 106 may optionally include a visual aid or marking 400 for indicating a location along the length of the lead 106 where the lead 106 should be positioned longitudinally with the proximal end 332 of the sheath 304 so that the distal end 344 of the lead 106 is properly positioned longitudinally with the distal end 312 of the sheath 304. By way of example and not limitation, the proximal lip 308 of the sheath 304 may include a marking 310 for assisting the surgeon in aligning the devices 106 and 304. In use, the surgeon may align the marking 400 with the corresponding marking 310 on the sheath 304 and then insert the combination of the sheath 304 and lead 106 into the patient P. Alternatively or in addition to the marking 400, as shown in FIG. 4B the lead 106 may include a detachable clip 404 that is biased, such as by a spring 408, to remain in place until removed, thereby preventing the lead 106 from being longitudinally advanced in a distal direction within the sheath 304 because the detachable clip 404 is blocked by the proximal lip 308 of the sheath 304. In yet another alternative to the marking 400, the lead 106 may include a detachable projection 412 for contacting the proximal lip 308 of the sheath 304, where the projection 412 physically prevents the lead 106 from being longitudinally advanced in a distal direction within the sheath 304 beyond the location of the proximal lip 308 because the projection catches on the proximal lip 308, thereby preventing further movement of the lead 106 within the sheath 304. In accordance with embodiments of the present invention, the projection 412 may include a frangible connection 416, thereby allowing the projection 412 to be easily removed so that the sheath 304 can be pulled out of the incision 126 over the lead 106 after the lead 106 is properly positioned. The projection 412 may also include a detent (not shown) or other feature for temporarily and detachably interlocking the lead 106 within the sheath 304. An audible click may optionally be used to further signal the surgeon or other personnel that the lead 106 is properly positioned within the sheath 304.

Referring again to FIG. 5A, the distal end 312 of the sheath 304 is shown with the lead 106 residing within the longitudinally oriented bore 340 of the sheath 304. When coupled with the lead 106, the rounded distal surface 324 of the sheath 304 provides protection to the tissue of the scrotal sac 134 for inserting the distal end 344 of the lead 106 into the scrotal sac 134.

Referring again to FIGS. 3-6, and in accordance with embodiments of the present invention, the sheath 304 includes at least one electrode exposure aperture 356, and more preferably, the sheath 304 includes a plurality of electrode exposure apertures 356 sufficient in number to correspond to the number of electrodes 118 located at the distal end 114 of the lead 106. The electrode exposure apertures 356 of the sheath 304 are preferably configured to substantially match the longitudinal position of the electrodes 118 of the lead 106. In accordance with embodiments of the present invention, the electrodes 118 of the electrode array 120 extend around the entire circumference of the lead 106. In accordance with embodiments of the present invention, the sheath 304 includes a plurality of electrode exposure apertures 356 spaced apart from the distal aperture 320, wherein the plurality of electrode exposure apertures 356 are longitudinally positioned along the length of the sheath 304 to correspond to the longitudinal position of the plurality of electrodes 118 of the electrode array 120 when the distal end 344 of the implantable lead 106 is located within the distal end 312 of the sheath 304. In addition, and as best seen in FIG. 6, embodiments of the present invention may comprise a plurality of apertures 356 for each electrode 118 of the electrode array 120. In accordance with embodiments of the present invention, electrode exposure apertures 356 may be axially aligned. That is, as shown in FIG. 6, electrode exposure apertures 356a are axially aligned along a first side of the sheath 304, electrode exposure apertures 356b are axially aligned along a second side of the sheath 304, and electrode exposure apertures 356c are axially aligned along a third side of the sheath 304, and so on. Alternatively, the plurality of electrode exposure apertures 356 may not be axially aligned (not shown in the drawings). Thus various configurations, numbers, sizes, and orientations of the electrode exposure apertures are possible, and such variations are within the scope of the present invention.

The electrode exposure apertures 356 advantageously allow the electrical stimulation system 102 to be tested prior to removal of the sheath 304 because at least a portion of the surface area of the electrodes 118 are exposed to tissue of the scrotal sac 134 even though the sheath 304 remains in place over the lead 106. As can be appreciated by those skilled in the art, the number of attempts of inserting an electrode array in the scrotal sac should be limited. As such, the electrode exposure apertures 356 allow the system 102 to be tested prior to removal of the sheath 304 so that the sheath 304 and lead 106 may be adjusted if necessary during the implanting procedure without having to first remove the sheath 304. That is, without the electrode exposure apertures 356, to test the system 102 the surgeon would have to first insert an electrode array into the scrotum using some type of sheath, remove the sheath, test the system, and assuming the system failed, thereafter remove the electrode lead, reinsert the electrode lead into the sheath, and then reinsert the sheath and the electrode lead yet again into the tissue of the scrotal sac and repeat the testing process. Thus, the electrode exposure apertures 356 reduce the probability of having to repeat the process outlined above because the system 102 can be tested with sheath 304 in place, and if necessary, the sheath 304 can be adjusted ill location to find a proper position for addressing the patient's pain.

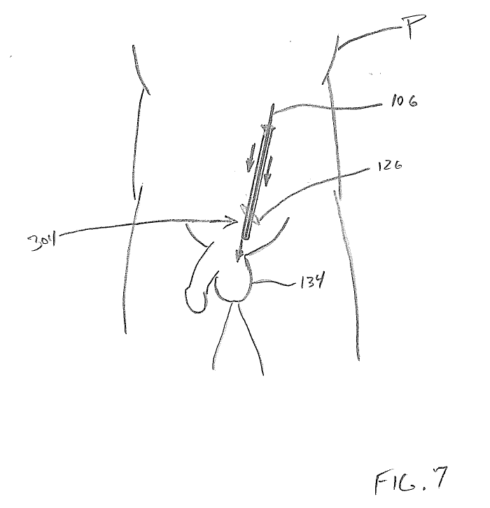

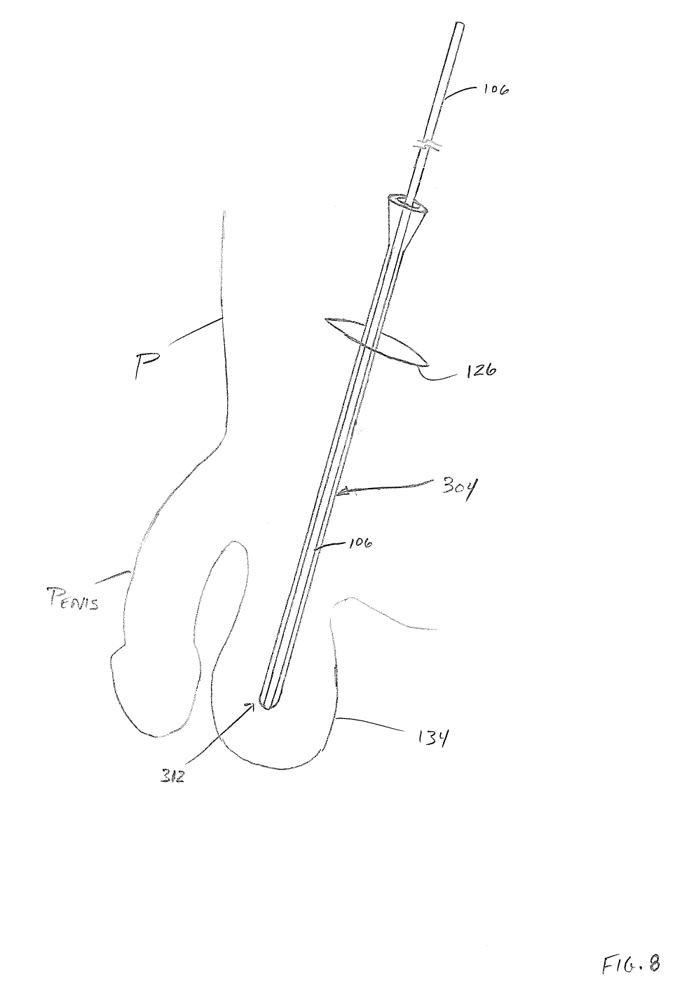

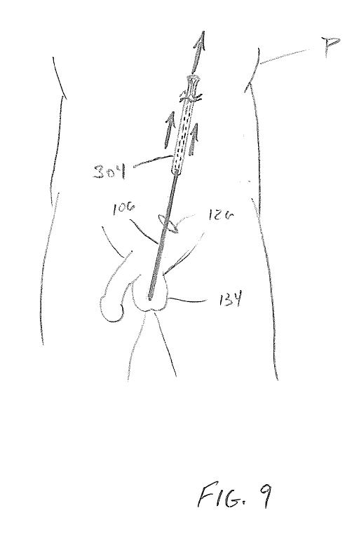

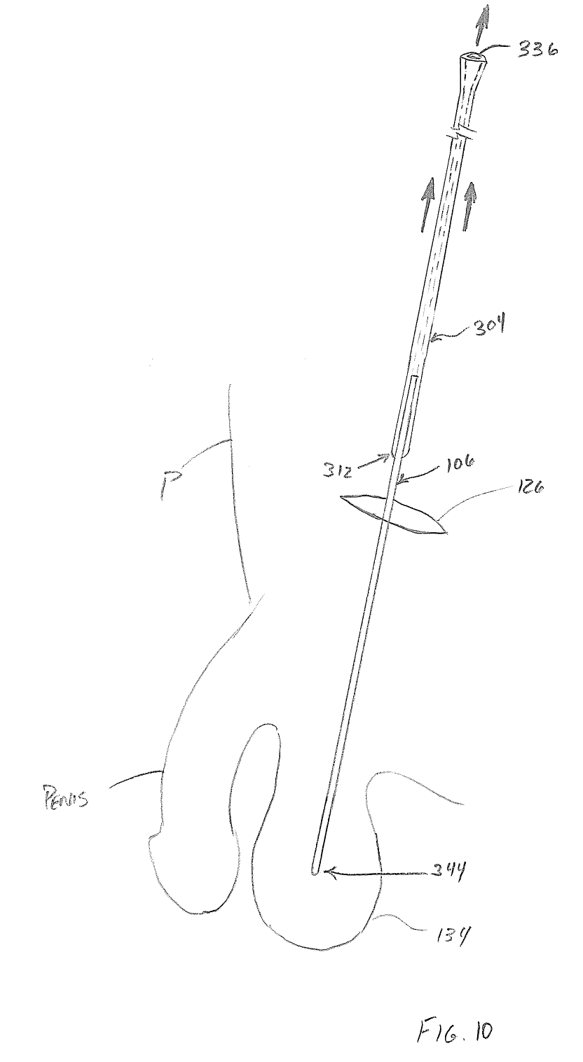

Referring now to FIG. 7, the implantation system 300 is shown in use to insert the electrodes 118 into the scrotal sac 134 of the patient P. In accordance with embodiments of the present invention, the lead 106 is first positioned within the sheath 304 prior to inserting the sheath 304 within the incision 126. Accordingly, the lead 106 is preferably inserted into the longitudinal oriented bore 340 of the sheath 304 such that the distal end 344 of the lead 106 is substantially aligned with the distal end 312 of the sheath 304, such as the substantially aligned distal ends 312 and 344 of the sheath 304 and lead 106 shown in FIGS. 5A and 5B. The sheath 304 and lead 106 combination are then inserted by the surgeon into the incision 126 arid advanced subcutaneously into the scrotal sac 134. The blunt tip 316 of the sheath 304 allows insertion of the distal end 344 of the lead 106 into the scrotal sac 134 without damaging the tissue of the scrotal sac 134. FIG. 8 shows the sheath 304 and lead 106 inserted into the scrotal sac 134. At this point, the surgeon may conduct a test to verify proper placement of the electrodes 118 within the scrotal sac. If necessary, the surgeon may adjust the location of the lead 106, such as be slightly withdrawing and/or advancing the implantation system 300 from its original position.

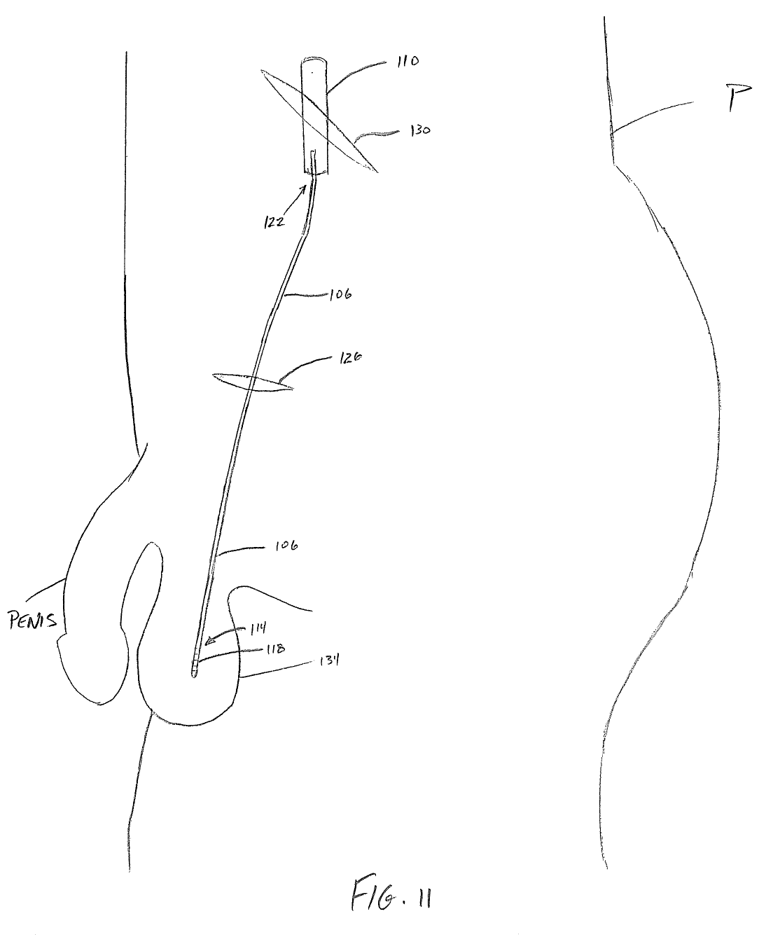

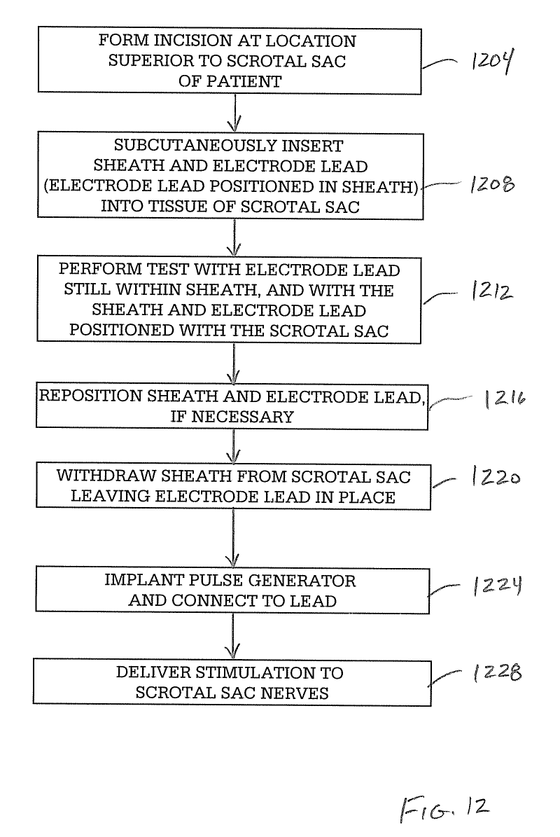

Referring now to FIGS. 9 and 10, after the distal ends 344 and 312 of the lead 106 and sheath 304, respectively, are satisfactorily positioned, the sheath 304 may be removed from the incision 126, thereby leaving the electrode 118 in place. Referring now to FIG. 11, the proximal end 122 of the lead 106 is preferably inserted subcutaneously between the first incision 126 to the second incision 130, where it is connected to the pulse generator 110. The incisions 126, 130 are then closed and the pulse generator 110 remotely controlled to adjust the nature of the electrical stimulation provided by the pulse generator 110 to the scrotal nerves located within the scrotal sac 134.

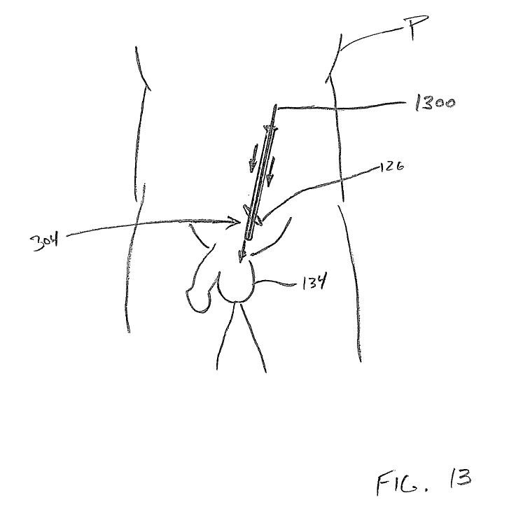

Referring now to FIG. 12, a flow diagram in accordance with embodiments of the present invention is depicted, the flow diagram directed to a method for implanting at least a portion of an electrical stimulation system in a scrotal sac of a patient. At step 1204 an incision 126 is made at a location anatomically superior relative to the scrotal sac 134 of the patient P. In step 1208 the sheath 304 and lead 106 are inserted through the incision 126, and advanced under the skin and into the scrotal sac 134 of the patient P. In step 1212, the optional step of testing the electrical stimulation system 102 is performed while the sheath 304 remains in place over the electrode 11 8 and at least a portion of the lead 106. Substeps may include adjusting the electrical current and asking the patient for feed back as to the effectiveness of the stimulation, where these substep are all performed with the sheath 304 remaining in place around the lead 106. In step 1216, the optional step of repositioning the sheath 304 and the lead 106 are performed. In step 1220 the sheath 304 is withdrawn from the scrotal sac 134 to leave the lead 106 and its one or more electrodes 118 positioned within the scrotal sac 134. In additional step 1224 comprises implanting the pulse generator 110 within the patient P and connecting the pulse generator 110 to proximal end 122 of the lead 106. This step may further include making an additional incision for the pulse generator 110, such as incision 130 shown in FIG. 1. Finally, in step 1228 stimulation is delivered to the scrotal sac nerves. Additional steps may include determining and adjusting the proper electrical stimulation signal. It is to be understood that the order of at least some of the steps set out above may be altered. For example, the pulse generator 110 could be implanted first, or repositioning may occur before a test is performed. Thus methods having a different order of steps than those shown in FIG. 12 or described herein are nonetheless within the scope of the present invention.

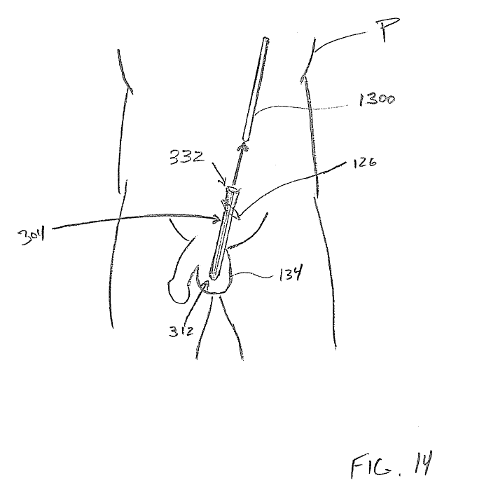

Referring now to FIGS. 13-15, and in accordance with embodiments of the present invention, the sheath 304 may be used in combination with a rod or introducer 1300 to insert the sheath 304 into the desired tissue (e.g., scrotal sac) prior to inserting the lead 106 into the sheath 304. More particularly, some procedures for placement of an electrode in tissue may require a less flexible or stiffer sheath 304. This can be provided by using a substantially solid introducer 1300, such as a relatively small diameter stainless steel rod that is loaded into the sheath 304 prior to inserting the sheath 304 through tissue. Although relatively small in diameter and somewhat flexible, the introducer 1300 in combination with the sheath 304 provides a blunt-tipped combination of structures such that resistance of tissue can be overcome when pushing or advancing the sheath into the targeted tissue.

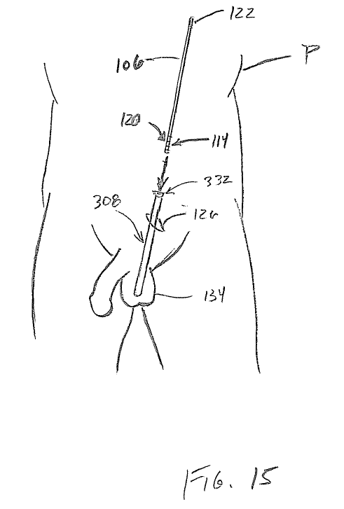

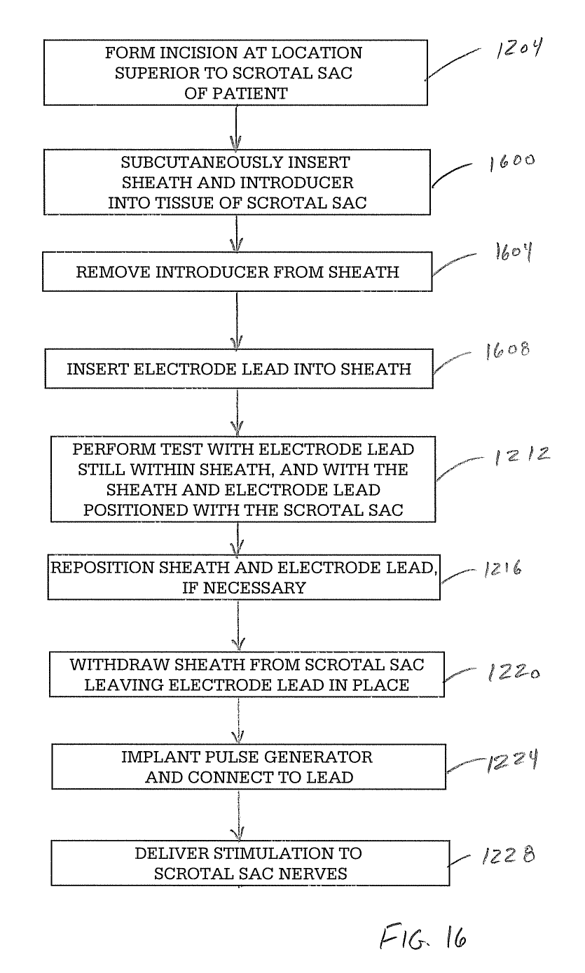

As shown in FIG. 13, the sheath 304 includes the introducer 1300 within its longitudinal bore 340 as the sheath 304 is placed into the incision 126 and advanced into the scrotal sac 134. After the combination of the sheath 304 and introducer 1300 are advanced into the proper location, the surgeon can withdraw the introducer 1300 from the sheath 304, as shown in FIG. 14. Referring now to FIG. 15, the lead 106 can then be inserted into the longitudinal bore 340 of the sheath 304 such that the distal end 344 of the lead 106 is substantially aligned with the distal end 312 of the sheath 304, such as the substantially aligned distal ends 312 and 344 of the sheath 304 and lead 106 shown in FIGS. 5A and 5B. The sheath 304 can the be adjusted in its location as previously described above. After the proper position for the electrode 118 is found, including potentially after testing the electrode 118 with the sheath 304 still in place, the sheath 304 can be removed from the surgical site leaving the lead 106 and electrode 118 implanted within the desired tissue, such as the tissue of the scrotal sac 134.

As those skilled in the art will appreciate, the combination of the sheath 304 and introducer 1300 may be inserted a portion of the distance to the targeted tissue, with the introducer 1300 then removed and the lead 106 inserted at that point, with the combination of the sheath 304 and lead 106 then advanced the remainder of the distance to the targeted tissue, such as the scrotal sac 134.

Referring now to FIG. 16, a flow diagram in accordance with embodiments of the present invention is depicted, the flow diagram directed to a method for implanting at least a portion of an electrical stimulation system in a scrotal sac of a patient. The method includes a number of steps as those shown in FIG. 12. At step 1204, an incision is made in the patient a location anatomically superior relative to the scrotal sac 134 of the patient P. At step 1600, the sheath 304 and introducer 1300 are inserted into the scrotal sac. At step 1604 the introducer 1300 is removed from the sheath 304, and at step 1608 the lead 106 is inserted into the hollow bore 340 of the sheath 304. Thereafter, the steps are similar to those shown in FIG. 12 for steps 1212 through 1228. Furthermore, other steps may include determining and adjusting the proper electrical stimulation signal, and again, it is to be understood that the order of at least some of the steps set out above may be altered. Thus methods having a different order of steps than those shown in FIG. 16 or described herein are nonetheless within the scope of the present invention.

Embodiments of the present invention further include a method of assembling an implantable system, the method comprising: providing an implantable pulse generator; providing an electrical lead; providing a sheath for holding the lead and enabling the lead to be implanted subcutaneously into the tissue (e.g., scrotum) of a patient; and inserting the lead into the sheath wherein the distal end of the lead resides within the distal end of the sheath. Additional steps of the method of assembling may include using a visual indicator to align the lead within the sheath, and/or using a removable projection or clip to prevent the lead from moving in a distal longitudinal direction once the lead in inserted into the sheath. The sheath may also include one or more apertures for exposing the electrode(s) of the lead, and additional methods steps may include aligning the one or more apertures with the electrodes. In accordance with embodiments of the present invention, a rod or introducer may also be provided, and the method may include preloading the introducer into the sheath.

The present invention has application for the treatment of intractable scrotal and/or testicular pain. In addition, the present invention has application for the treatment of other indications, including stimulation of the testicles. The present invention may also be used in combination with drug or fluid delivery systems, such as a system that delivers both electrical stimulation and drugs to the scrotum. Thus, the present invention may have other health, reproductive and/or sex related benefits, and such uses/applications are within the scope of the present invention.

Implantable portions of the neurostimulator 102 and/or its associated features may be made from one or more materials that possess the appropriate strength characteristics necessary to withstand conditions from the body and associated implants when used in medical applications. In addition, the materials may be chosen to provide desired flexibility characteristics. In accordance with embodiments of the present invention, examples of materials that may be used to make at least portions of the neurostimulator 102 include, but are not necessarily limited to, silicone, polyether ether plastics, such as ketone (PEEK), polyether ketone ketone (PEKK), ultra high molecular weight polyethylene (UHMWPE), and polymethylmethacrylate (PMMA); metals, such as titanium and stainless steel; composites; as well as other tissue compatible materials.

In accordance with at least one embodiment of the present invention, the sheath may be made of an absorbable material such as polyglycolide or polyglycolic acid. Here, the sheath is implanted with the electrode and closed within the surgical site, where subsequently the sheath is absorbed by the tissue leaving the electrode in place. In accordance with another embodiment of the present invention, the electrode lead may comprise an enlarged diameter comprising an absorbable material coating over some or portions of the electrode lead, wherein the coated electrode lead includes a blunt tip sized to eliminate or mitigate damage to tissue as the electrode lead is being positioned within the tissue of the patient.

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

While particular embodiments of the present invention have been described in some detail, it should be understood that other related embodiments are intended to be within the scope of the present invention. For example, the sheath 304 may comprise structure different than that shown in the figures to provide other ways to functionally and structurally provide an implantable electrode within the scrotal sac of a male. The present invention may also be used to provide electrical stimulation to the scrotal sac for purposes other than treating pain. In addition, the present invention may be used in veterinary medicine. Such modifications, alternate uses and/or adaptations are within the scope of the present invention, including those that are well within the knowledge of those of ordinary skill in this art, regardless of whether such structures and/or uses are conventional or developed in the future.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

While various embodiments of the present invention have been described in detail, it is apparent that modifications and adaptations of those embodiments will occur to those skilled in the art. However, it is to be expressly understood that such modifications and adaptations are within the spirit and scope of the present invention, as set forth in the following claims.

What is claimed is:

1. A device used for implanting an implantable lead into a patient, the implantable lead having a distal lead end and a proximal end wherein the proximal end of the implantable lead is operatively associated with an implantable pulse generator, the implantable lead including at least one electrode wherein the electrode is for placement into body tissue fo the patient, the system comprising:
a sheath comprising a distal end having a blunt tip in proximity to a distal aperture, the sheath further comprising a longitudinally oriented bore adapted for receiving temporarily at least a portion of the implantable lead including the electrode, the longitudinally oriented bore extending between the distal aperture of the sheath and a proximal aperture at a proximal end of the sheath, the sheath having at least one electrode exposure aperture corresponding to a longitudinal position of the electrode;
wherein the distal lead end of the implantable lead cooperates with the blunt tip of the sheath for allowing advancement of the electrode into the tissue of the patient.

2. The device as claimed in claim 1, wherein the sheath may be withdrawn from the tissue to leave the electrode positioned within the tissue.

3. The device as claimed in claim 1, wherein the tissue is a scrotum and the longitudinally oriented bore has a length shorter than a length of the implantable lead.

4. The device as claimed in claim 1, wherein the implantable lead comprises at least one of a marking, projection, and clip for longitudinally positioning the implantable lead relative to the sheath

5. The device as claimed in claim 1, wherein the electrode resides within an electrode array, the electrode array comprising a plurality of electrodes.

6. The device as claimed in claim 5, wherein the sheath comprises a plurality of electrode exposure apertures corresponding to the position of the electrodes of the electrode array.

7. The system as claimed in claim 6, wherein the plurality of electrode exposure apertures are longitudinally positioned along a length of the sheath to include the longitudinal positions of the plurality of electrodes of the electrode array when the distal end of the implantable lead is located within the distal end of the sheath,

8. The system as claimed in claim 7, wherein the plurality of electrode exposure apertures are circumferentially distributed around the sheath at each electrode of the electrode array.

9. The system as claimed in claim 8, wherein at least one electrode exposure aperture at a first longitudinal position along the sheath is axially aligned with at least another electrode exposure aperture at different longitudinal position along the length of the sheath.

10. A system for implantation and electrical stimulation of a patient having a scrotal sac, the system comprising:
an implantable pulse generator;
an implantable lead operatively associated with the pulse generator;
at least one implantable electrode operatively associated with the implantable lead; and
means for introducing the implantable electrode subcutaneously into the scrotal sac.

11. The system as claimed in claim 10, wherein the means for introducing comprises a sheath comprising a distal end having a blunt tip and a distal aperture, the sheath further comprising a longitudinally oriented bore extending through at least a portion of the sheath and contiguous between the distal aperture and a second aperture spaced apart from the first aperture.

12. The system as claimed in claim 10, wherein the sheath includes at least a third aperture spaced apart from the distal aperture, wherein the third aperture is longitudinally positioned along a length of the sheath to correspond to the longitudinal position of the at least one implantable electrode when the distal end of the implantable lead is located within the distal end of the sheath.

13. The system as claimed in claim 10, wherein the at least one implantable electrode resides within an electrode array, the electrode array comprising a plurality of electrodes, and wherein the sheath includes a plurality of electrode exposure apertures spaced apart from the distal aperture.

14. The system as claimed in claim 10, wherein the implantable lead comprises means for limiting longitudinally movement within the means for introducing.

15. The system as claimed in claim 14, wherein the means for limiting longitudinal movement comprises one or more of a frangible projection and a removable clip.

16. The system as claimed in claim 10, wherein the means for introducing comprises a means for passing an electrical current while the implantable electrode resides with in the means for introducing.

17. The system as claimed in claim 16, wherein the means for passing an electrical current comprises one or more electrode exposure apertures.

18. A method for implanting at least a portion of an electrical stimulation system in a patient, the method comprising:
(a) causing an incision to be made in the patient, the incision spaced apart from an implant target location;
(b) inserting a distal end of a sheath through the incision, the sheath holding at least a portion of an implantable lead having an electrode;
(c) advancing a blunt tip of the sheath subcutaneously to and into a tissue of the patient to the implant target location, the sheath having a longitudinally oriented bore for holding and selectively positioning the electrode into the tissue, wherein the sheath and electrode are advanced together during the advancing step; and
(d) withdrawing the sheath from the tissue while maintaining the electrode within the tissue.

19. The method as claimed in claim 18, further comprising inserting the implantable lead into the sheath until a distal end of the implantable lead aligns substantially proximate the distal end of the sheath.

20. The method as claimed in claim 18, wherein the at least one electrode resides within an electrode array, the electrode array comprising a plurality of electrodes.

21. The method as claimed in claim 20, further comprising removing at least one of a clip and a frangible projection from the electrical lead prior to the withdrawing step.

22. The method as claimed in claim 18, wherein the withdrawing step comprises pulling the sheath such that the distal end of the sheath passes over the at least one electrode.

23. The method as claimed in claim 18, wherein the tissue is associated with a scrotal sac of the patient.

24. A method for treating intractable scrotal and/or testicular pain in a patient having a scrotal sac, the method comprising:
causing an incision to be made in the patient, the incision spaced apart from the scrotal sac;
inserting a sheath and an electrode lead having at least one electrode through the incision, the sheath comprising a distal end that includes a blunt tip, the sheath having a longitudinally oriented bore holding at least a portion of the electrode lead, wherein the sheath extends longitudinally in a distal direction beyond the electrode;
advancing the blunt tip of the sheath subcutaneously to and into the scrotal sac;
positioning the distal end of the electrode lead within the scrotal sac;
withdrawing the sheath from covering the electrode while maintaining the electrode within the scrotal sac;
implanting a pulse generator into the patient;
connecting a proximate end of the electrode lead to the pulse generator; and
causing an electrical pulse to be generated by the pulse generator, wherein the electrical pulse passes through the electrode lead and to the electrode located in the scrotal sac.

25. The method as claimed in claim 24, further comprising testing the electrode before the step of withdrawing.

26. The method as claimed in claim 25, further comprising moving the sheath and the electrode after the step of testing and before the step of withdrawing.

27. The method as claimed in claim 25, further comprising adjusting the electrical pulse generated by the pulse generator before the step of withdrawing.

29. The method as claimed in claim 24, further comprising removing at least one of a clip and a frangible projection from the electrical lead prior to the withdrawing step.

30. A method of assembling an implantable electrical stimulation system, the method comprising:
providing an implantable pulse generator;
providing an electrical lead comprising at least one electrode; and
providing a sheath for holding the lead, wherein the sheath comprises a diameter sized for receiving the lead;
wherein a distal end of the lead is adapted for residing within a distal end of the sheath.

31. The method as claimed in claim 30, wherein the sheath comprises at least one aperture corresponding to a longitudinal location of the electrode when the distal end of the lead is situated within the distal end of the sheath.

32. The method as claimed in claim 30, further comprising providing an introducer, wherein the sheath comprises a diameter sized for receiving the introducer.

33. The method as claimed in claim 30, wherein the lead comprises a removable projection or clip to prevent the lead from moving in a distal longitudinal direction once the lead in inserted into the sheath.
